(19) United States
(12) Patent Application Publication
(10) Pub. No.: US 2005/0192228 A1
(43) Pub. Date: Sep. 1, 2005

(54) Tripeptide derivatives for the treatment of neurodegenerative diseases

(76) Inventors: Jean Rapin, Paris (FR); Hans Klaus Witzmann, Egglkofen (DE); Jean-Marie Grumel, Tassin la Demi-Lune (FR); Jacques Gonella, Muttenz (CH)

Correspondence Address: Sherry M. Knowles, Esq.;KING & SPALDING, LLP, 45th Floor, 191 Peachtree Street, N.E., Atlanta, GA, 30303, US

(21) Appl. No.: 10/635,696

(22) Filed: Aug. 5, 2003

Related U.S. Application Data
(63) Continuation of application No. PCT/EP02/01181, filed on Feb. 5, 2002.

(30) Foreign Application Priority Data
Feb. 5, 2001 (DE) 10 105 039.9

(51) Int. Cl. A61K 38/05; C07K 7/06

(52) U.S. Cl. 514/18; 530/331

(57) ABSTRACT

The invention relates to the use of specific tripeptides for the treatment of neurodegenerative diseases. The tripeptide derivatives satisfy the following formula (I): wherein X represents OH, (C₁₋₅)alkoxy, NH₂, NH—C₁₋₅-alkyl, N(C₁₋₅alkyl)₂; R₁ is a residue derived from any of the amino acids Phe, Tyr, Trp, Pro, each of which may optionally be substituted by a (C₁₋₅)alkoxy group, a (C₁₋₅)alkyl group or a halogen atom, and Ala, Val, Leu, or Ile; R₂ is a residue which is derived from any of the amino acids Gly, Ala, Ile, Val, Ser, Thr, His, Arg, Lys, Pro, Glu, Gln, pGlu, Asp, Leu and Asn; R₃ and R₄ independently represent H, OH, (C₁₋₅)alkyl, or (C₁₋₅)alkoxy, provided that R₃ and R₄ are not both OH or (C₁₋₅)alkoxy; R₅ represents H, OH, (C₁₋₅)alkyl or (C₁₋₅)alkoxy; and wherein R₀ preferably represents a cinnamoyl residue; or pharmaceutically acceptable salts thereof.

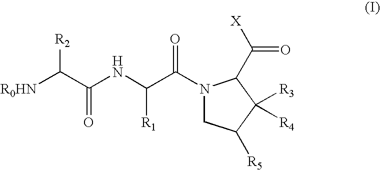

The invention relates to the use of tripeptide derivatives for the treatment of neurodegenerative diseases, particularly those caused by apoptotic processes.

### BACKGROUND ART

Neurodegenerative diseases are characterised by a degradation or degeneration of nerves which are generally caused by apoptosis. Examples of neurodegenerative diseases include Alzheimer's disease, mild cognitive impairment, Parkinson's disease as well as AIDS-related neurological disorders. For example, in Alzheimer's disease, the nerve degradation leads to a disruption of the ability to remember, speak, think and make decisions. The reasons for these disorders are not known in detail. On the biochemical level, a change in the cortical cholinergic systems with a decrease in the formation of the neurotransmitter acetylcholine is detectable. In the cerebral cortex of patients suffering from Alzheimer's disease, the acetylcholine concentration is decreased by 20 to 40%. As a result thereof, nerve ends are attacked, and this leads ultimately to the death of the cerebral cells, particularly those of the hippocampus.

Clinically, Alzheimer's disease is characterized by three distinct phases: a phase of pre-dementia, a phase of light dementia, and a phase of severe dementia. In the phase of pre-dementia, a neuronal degeneration is observed, particularly on the level of the hippocampus. Later on, the typical amyloid deposition occurs.

Classical treatments of Alzheimer's disease using nootropic substances only alleviate the symptoms of the disease, particularly cognitive disorders, during administration of the substance. Once the administration is discontinued, the symptoms reappear. This is in contrast to an anti-neurodegenerative effect in the hippocampus which results in a decrease and preferably stopping of the evolution of the hippocampal neuron degeneration. Nootropic substances are for example disclosed in EP 0 316 218 B1.

Recent therapeutic approaches for Alzheimer's disease therefore address the stabilisation of the acetylcholine concentration, particularly by inhibiting acetylcholine esterase which degrades acetylcholine to acetate and choline. However, the use of acetylcholine esterase inhibitors shows the drawback that this results in an only temporary improvement which is not suitable for stopping or even reversing the nerve degeneration.

On the other hand, so-called neurotrophic factors or neurotrophines are known to which a significant influence on the survival, growth and differentiation of discrete neuronal populations is ascribed. The neurotrophine family includes nerve growth factor (NGF), brain derived neurotrophic factor (BDNF), neurotrophine-3 (NT-3), neurotrophine-4 (NT-4) and the CNTF-family (ciliary neurotrophic factor). Neurotrophines are small basic proteins with a molecular weight of 26 to 28 kDa. NGF is the best characterised member of the neurotrophine family which shows activity in many different tissues.

In the peripheral nervous system (PNS) NGF is critical to the development of sympathetic and certain sensory nerves. In the central nervous system (CNS), NGF serves a trophic role in the development and maintenance of cholinergic neurons of the basal forebrain. It also plays a role in adult CNS tissues in neuronal regeneration.

It is known that cholinergic neurons produce acetylcholine in the presence of NGF rather than in its absence. Moreover, it has also been demonstrated that the administration of NGF to primates leads to the regeneration of cholinergic cell bodies. Based on this finding it is assumed that an altered activity of NGF may thus be a starting point for the degeneration of cholinergic neurons. At least theoretically it thus appears that neurotrophic substances are suitable for the treatment of neurodegenerative diseases such as Alzheimer's disease. However, these physiologically occurring substances have a short action radius similar to autocrine or paracrine substances. Therefore it is until today not possible to use common therapeutical routes (enteral or parenteral) for their application, as they are processed proteolytically in the blood circulation and other tissues and are thereby inactivated. Besides, it is known that they do not pass the blood-brain barrier (BBB) which is a prerequisite of CNS activity.

Clinical trials carried out with recombinant human neurotrophines have failed so far. A conceivable intracerebral administration should be excluded by practical consideration. Therefore, a transfer of results from in vitro experiments with NGF or other neurotrophines, as well as with fragments of these peptides, to a possible therapeutic application is not possible.

### SUMMARY OF THE INVENTION

Hence, it is the object of the present invention to provide specific substances which lead to a stopping and preferably reversing of nerve degeneration, particularly of hippocampal cells, and which are also suitable for common therapeutic administration thus allowing their use as medicament for the treatment of neurodegenerative diseases.

This object of the present invention is solved by the use of compounds of the following formula (I): 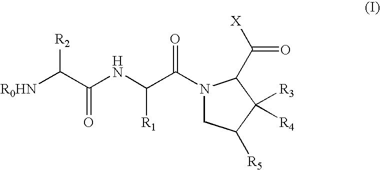
wherein X represents OH, (C₁₋₅)alkoxy, NH₂, NH—C₁₋₅-alkyl, N(C₁₋₅alkyl)₂;
R₁ is a residue derived from any of the amino acids Phe, Tyr, Trp, Pro, each of which may optionally be substituted by a (C₁₋₅)alkoxy group, a (C₁₋₅)alkyl group or a halogen atom, and Ala, Val, Leu, or Ile;
R₂ is a residue which is derived from any of the amino acids Gly, Ala, Ile, Val, Ser, Thr, His, Arg, Lys, Pro, Glu, Gln, pGlu, Asp, Leu and Asn;
R₃ and R₄ independently represent H, OH, (C₁₋₅)alkyl, or (C₁₋₅)alkoxy, provided that R₃ and R₄ are not both OH or (C₁₋₅)alkoxy;
R₅ represents H, OH, (C₁₋₅)alkyl or (C₁₋₅)alkoxy;
and wherein R₀ represents a group of the formula 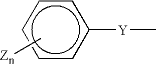
   wherein Y represents —CO—, —CH₂CO—, —CH₂CH₂CO—, —CH₂CH₂CH₂CO—, —CH═CH—CO or —OCH₂CO—, and wherein Z represents a halogen atom, a trifluormethyl group, (C₁₋₄) alkoxy group, (C₁₋₄)alkyl group; or wherein two neighbouring substituents may form a (C₁₋₃)alkylendioxy group; and
wherein n is 0 or an integer of from 1 to 5;
   or pharmaceutically acceptable salts thereof;
for the preparation of a medicament useful in the treatment of neurodegenerative diseases.

### DETAILED DESCRIPTION

If not indicated otherwise, the amino acid residues may be present both in the D-form as well as the L-form, the L-form being preferred.

Preferred are compounds of the formula (I) in which R₁ is a residue derived from the amino acid Ile or one of the amino acids Phe, Tyr, Trp, which each may be optionally substituted with one or more (C₁₋₅)alkoxy groups, (C₁₋₅)alkyl groups or one or more halogen atoms, particularly a residue which is derived from Ile or Phe which is optionally substituted with one or more (C₁₋₅)alkoxy groups, (C₁₋₅)alkyl groups or one or more halogen atoms.

In formula (I), X is preferably (C₁₋₅)alkoxy, NH₂, NH—(C₁₋₅) alkyl or N(C₁₋₅alkyl)₂, more preferred are NH₂, NH(C₁₋₃) alkyl and N(C₁₋₃ alkyl)₂.
R₂ is preferably a residue derived from the amino acid Gly or Ile.
R₃ and R₄ preferably independently from each other represent H, (C₁₋₅)alkyl or (C₁₋₅)alkoxy, provided that R₃ and R₄ are not (C₁₋₅)alkoxy, more preferred are H, (C₁₋₃)alkyl or (C₁₋₃)alkoxy.
R₅ preferably represents H, (C₁₋₅)alkyl or (C₁₋₅)alkoxy, particularly preferred are H, (C₁₋₃)alkyl or (C₁₋₅)alkoxy. R₀ is preferably a cinnamoyl residue.

For particularly preferred compounds of formula (I), R₀ is preferably a cinnamoyl residue, R₁ is a residue which is derived from Phe which is optionally substituted with one or more (C₁₋₅)alkoxy groups, (C₁₋₅)alkyl groups or one or more halogen atoms, or which is derived from the amino acid Ile, R₂ is a residue derived from the amino acid Gly or Ile, R₃, R₄ and R₅ represent a hydrogen atom, X is NH₂, NH—(C₁₋₃) alkyl or N(C₁₋₃ alkyl)₂.

For particularly preferred compounds of formula (I), R₁ is a residue which is derived from Phe which is optionally substituted with one or more (C₁₋₅)alkoxy groups, (C₁₋₅) alkyl groups or one or more halogen atoms, or which is derived from the amino acid Ile, R₂ is a residue derived from the amino acid Gly or Ile, R₃, R₄ and R₅ represent a hydrogen atom, X is NH₂, NH—(C₁₋₃)alkyl or N(C₁₋₃ alkyl)₂, and Y₁ and Y₂ independently from each other represent H or (C₁₋₃)alkyl.

R is preferably a cinnamoyl residue.

Most preferred compounds of formula (I) are cinnamoyl-glycyl-L-phenylalanyl-L-prolineamide, cinnamoyl-isoleucyl-phenylalanyl-L-proline ethylamide, cinnamoyl-isoleucyl-isoleucyl-prolineamide, or a pharmaceutically acceptable salt thereof.

The abbreviations used for the amino acids (Phe for phenylalanine etc. as well as partially the one-letter-codes used below, such as F for phenylalanine) are known to the skilled person (see e.g. Beyer and Walter, Lehrbuch der Organischen Chemie, 21st edition, S. Hirzel Verlag Stuttgart 1988). Hence, Phe means phenylalanine, Gly glycine etc. The expression “a residue derived from the amino acid Phe” thus means a benzyl (—CH₂—C₆H₅) residue. Accordingly, “a residue derived from the amino acid Gly” means a hydrogen atom, “a residue derived from the amino acid Ala” a methyl group etc.

The synthesis of the tripeptide derivatives used according to the present invention is not particularly limited and can be carried out according to known methods, preferably stereo-specific processes of peptide chemistry in which the L- or D-configuration of the respective amino acids or their derivatives is maintained. Particularly suitable are the syntheses disclosed in EP 0 316 218 B1.

The compounds of formula (I) used according to the present invention are lipophilic substances and suitable for enteral and in appropriate formulations for parenteral administration.

An administration in a dose of 1 to 5 mg per kilogram bodyweight per day, preferably 75 to 375 mg per day is usually effective. To achieve the anti-neurodegenerative effect, an administration over several days (for example at least 4 or 5 days) is generally preferred.

The tripeptide derivatives to be used according to the present invention show a very low toxicity. In mice, using dosages of up to 1000 mg/kg p.o. according to the Irwin test, no lethal or cramp causing effects were observed.

The tripeptide derivatives may be used for the production of pharmaceutical compositions which are suitable for administration in different ways, e.g. parenteral (intravenous, intramuscular, subcutane), via the respiratory tract (buccal, sublingual, nasal, bronchial), the transdermal route (percutane) and the enteral route (peroral).

The pharmaceutical compositions of the present invention further contain a pharmaceutically acceptable excipient, pharmaceutically acceptable diluents or adjuvants. Standard techniques may be used for their formulation, as e.g. disclosed in Remington's Pharmaceutical Sciences, 20^{th} edition Williams&Wilkins, PA, USA.

The administration form is selected depending on the administration route and comprises inter alia tablets, capsules, powders and solutions.

For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives. Preferred are formulations containing 75 to 225 mg, more preferably 100 to 200 mg, of the tripeptide derivate per administration unit, e.g. per tablet or capsule.

A particularly preferred formulation for oral administration is a coated tablet containing 100 mg Cinnamoyl-Gly-Phe-ProNH₂ as well as microcristalline cellulose, maize starch, Povidon 25: Crospovidon, Macrogol 4000, titanium dioxide (E171), and ferric oxide (E172).

For parenteral administration, sterile ethanol-containing aqueous solutions are preferred. Suitable sterile aqueous solutions or physiological saline solution may contain 10% v/v ethanol. A volume of 10 ml of such a solution is used to dissolve 100 mg of lyophilised Cinnamoyl-Gly-Phe-ProNH₂, in an appropriate medical device for injection.

The anti-neurodegenerative effect of the tripeptide derivatives to be used according to the present invention is surprising, particularly when administered parenterally or enterally. Although the nootropic effect of these substances is known from EP 0 316 218 B1, the finding that these substances do not only show a temporary nootropic effect during administration, but a stopping of nerve degeneration could not be expected.

The administration of the substances to be used-according to the present invention is preferred for the treatment of Alzheimer's disease, and particularly mild cognitive-impairment.

The superior therapeutic properties of the tripeptide derivatives used according to the present invention will be further illustrated below using particularly useful models for Alzheimer's disease. Using these models, it could be demonstrated that the administration of the tripeptide derivatives used according to the present invention does not only result in an increase of the number of hippocampus neurons, but also results in an improvement of the learning behaviour of the rats used in the tests.

Experiments

1. Introduction

There are no animal models for Alzheimer's disease. The transgenic mouse model is only of limited use as far as behavior is concerned. Therefore we present a battery of three rat models. Each model reproduces one of the physiopathologic features of the disease: neurofibrillary degeneration in the vincristine model, degeneration by beta-amyloid in the Gp120 model, and apoptosis in the dexamethasone model.

Vincristine is an anticarcinogen that is used as a synchronizing agent. This molecule binds to the spindle of microtubules, thus blocking the cellular multiplication during the metaphase. It is a spindle poison. The neurons do not multiply under physiologic conditions, but the axons are made of neurofibrils whose structure is similar to the one of the microtubules of the spindle. Vincristine binds to these neurofibrils thus causing peripheral neural conduction disorders in patients who are treated for neoplasm. These effects mainly affect the white matter of the axons. As vincristine does not pass the blood-brain barrier it has to be given by intracerebroventricular administration. The repeated ICV administration of vincristine causes a degeneration of the conduction pathways with the appearance of abnormal neurofibrils similar to those observed in Alzheimer's disease. This degeneration mainly affects the periventricular structures. The hippocampi are affected through a decrease of their ramifications, while the cell bodies of the neurons are not altered. We also showed that the repeated administration of vincristine is only possible for a short time (5 days at a maximum). After this period, a protective layer forms on the glia cells that prevent the diffusion of vincristine from the CSF to the neighboring nerve cells. There is no spontaneously developing Alzheimer's disease in rats that would show the different types of degenerative lesions (beta-amyloid plaques, neurofibrillary and vacuolar degeneration). ICV administration of vincristine, which may cause neurofibrillary degeneration, may be used as a partial animal model of this disease. The studies that we conducted on rats in our laboratory showed a decrease of hippocampal arborization. After the administration of vincristine, the learning ability of the animals is diminished, while a neurofibrillary degeneration of the hippocampus is observed. As it is difficult to evaluate this degeneration in a screening model, the only evaluation parameters are the learning capacities. This model has been standardized and validated in our laboratory to obtain a good reproducibility.

Dexamethasone is a corticoid similar to natural cortisol. Stress causes a release of catecholamine and cortisol (corticosterone in rats). Repetitive stress—“chronic stress”—leads to a down regulation of the hippocampal receptors to glucocorticoides. These studies, which first started in rats, were confirmed in human beings, where a specific form of depression (Golf war syndrome) is observed at first, then followed by an irreversible impairment of cognitive capacities. MRI studies on human beings showed a degeneration of the hippocampal neurons. A similar syndrome is observed in diseases associated with hypercorticism (Morbus Cushing) or during long-term corticoid treatment (Multiple Sclerosis). In rats, the repeated administration of dexamethasone first causes a down regulation of the type II receptors of the hippocampus that is followed by a degeneration of the neurons of the CAII and CAIII layers. The learning ability of the animals is impaired. This model has been standardized and validated in our laboratory to obtain a good reproducibility.

Gp120 is a surface glycoprotein of the HIV virus. This glycoprotein plays a fundamental role in the binding of the virus and the passage of viral RNA in the host cell. Isolated Gp120 also binds to CD4 receptors of the cells and causes a lesion of the membranes with the appearance of the pores into which the calcium ions dive, thus causing cellular apoptosis. On the surface of the cells Gp120 forms a deposit of glycated proteins that are quite similar to the beta-amyloid deposits. The HIV virus does not pass the blood-brain barrier, but the virally infected macrophages pass it easily. Once arrived in the CNS, the macrophage dies and releases its cellular contents with the Gp120 and other viral constituents. This Gp120 binds to the CD4 of the glia cells and also to the neurons, thus causing complex dementia as the final stage of AIDS. The observed lesions are similar to those of Alzheimer's disease, but in a different distribution. In Alzheimer's disease the cholinergic structures of the nucleus of the basis and the limbic system are the first altered, whereas in complex dementia of AIDS the distribution of the plaques is general. We have conducted several rat studies using this Gp120 model. The administration into the cerebrospinal fluid causes an important decrease of the neurons in the hippocampi associated with a decrease in the regional consumption of glucose and a decrease in blood flow. The degeneration is observed in the CAII and CAIII layers of the hippocampus that induces an impairment of the learning capacities. This model has been standardized and validated in our laboratory to obtain a good reproducibility.

2. Materials and methods

2.1 Animals

Male Wistar rats (Charles River, Saint Aubin les Elbeuf, France), each weighing on average 280-300 grams, were used in the animal experiments. For a period of one week, the animals were placed in stablings in an animal laboratory where the following parameters were controlled
day/night rhythm 7.00 a.m./7.00 p.m.
temperature: 22+/−1° C.
hygrometry: 50+/−10%
   The animals got drinking water and a standard feed UAR A03 ad libitum.
   2.2 Gp120 Model

40 rats were slightly anesthetized by ether, an incision was made into the skin of the skull, and the skull was pierced by means of a dental burr. A metal needle was stereotaxically directed to the lateral ventricle, and then fixed by means of dental cement. Every day of the experiment, the patency of the needle was controlled.

Three days after introducing the needle (recovery from the postoperative shock) the non-anesthetized animals were divided into 2 groups:
one group of 10 rats to which 5 μl of physiological serum was injected
one group of 30 rats to which 5 μl of physiological serum containing Gp120 in an amount of 10 nM/kg was injected

The laboratory experiments showed that the degeneration depended on the dose of Gp120; the dose of 10 nM/kg/day causes a 40 to 60% loss of the hippocampal neurons. This administration was repeated every day for a period of five days.

Ten days after the last administration of Gp120, the thirty rats were randomly divided into three groups of 10 animals each:
(i) one group of 10 rats received 1 ml/kg/day of normal saline (controls) for a period of 5 days.
(ii) one group of 10 rats received 10 mg/kg/day of Cinnamoyl-Gly-L-Phe-L-PrONH₂ dissolved in normal saline for a period of 5 days.
(iii) one group of 10 rats received 10 mg/kg/day of tacrine peros for a period of 5 days

The injections were given at 9.00 a.m.

The first group of 10 rats (without Gp120 administration) received 1 ml/kg water under the same conditions.

During the last five days of treatment, the rats were placed under the common learning conditions of the three models between 10.00 a.m. and 11.00 a.m. (see below).

2.3 Dexamethasone Model

40 rats were kept and operated as described above. Then, they were divided into the following groups:
one group of 10 rats which received 5 μl/day of physiological serum
one group of 30 rats which received 50 mg/kg/day dexamethasone, dissolved in physiological serum.

During the last five days of treatment, the rats were placed under the common learning conditions of the three models between 10.00 a.m. and 11.00 a.m. (see below).

After the last learning session at 10.00 a.m., the rats were sacrificed by means of decapitation, and the hippocampi were quickly removed and put on a plate cooled to 0° C. The number of glucocorticoid receptors was determined by a binding method using a labeled corticoid and a specific inhibitor to the binding of the corticoid to the receptor (here a total agonist). The protein content was measured by Lowry's method.

The hippocampi of each rat were homogenized in 2 ml of sodium EDTA Glycerol molybdate buffer. The homogenate was centrifugated at 100.000 g for 60 minutes. An aliquot of the supernatant was diluted in distilled water, and the protein content was measured by Lowry's method. This protein concentration was between 1.3 and 1.7 mg/ml.

The rest of the supernatant was divided into three parts of 0.2 ml each. Increasing concentrations (25, 50 and 75 nmoles/ml) of dexamethasone 3H (Amersham 50 Ci/mM) were added to these parts. Three other preparations were carried out under the same conditions, while a saturating quantity of a total antagonist of the receptors (RU 28362) was added to obtain a non-specific binding of the labeled dexamethasone.

After one night of incubation at 4° C., charcoal/dextran was added to absorb the proteins and the bound dexamethasone. After centrifugation, the radioactivity of the supernatant was measured by liquid scintillation.

The radioactive quantities of the dexamethasone bound to the protein content are recorded. Results are expressed in femtomoles of the labeled corticoid bound to the receptor per mg of proteins.

2.4 Vincristine Model

Rats were kept and treated as described above in 3. Then they were divided into the following groups:
one group of 10 rats which received 5 μl/day of physiological serum over a period of 5 days
one group of 30 rats which received 5 μl/day of physiological serum containing 5 μg/kg vincristine over a period of 5 days

The experiments showed that the degeneration depends on the vincristine dosage. A dosage of 5 μg/kg/day causes a 40 to 60% loss of the hippocampal arborization.

After the last administration, the second group of 30 rats was divided at random into three sub-groups and treated as described above in 3.

2.5 Learning

During the last five days of treatment, at 10.00 a.m. (1 hour after the administration of Cinnamoyl-Gly-Phe-PrONH₂), the animals were placed into a learning cage for sound avoidance conditioning (Conditioning avoidance response). The animals learned to climb a pole first to escape from and then to avoid an electric shock. This method was standardized and quantified. (see Le Poncin M., Lafitte J. C., Rapin J. R., Sound Avoidance Conditioning and a Mathematical approach to the description of acquisition performance, Math. Biosciences 59 (1982) 242-268).

For a period of five days, the animals were placed everyday under these learning conditions for a total of ten tests a day. Results are expressed as percentage of adequate responses, and the kinetics of the responses is represented by a multi-exponential maximum curve.

In all experiments, which we conducted in the laboratory, all control animals showed an avoidance response on Day 5 of learning (100% adequate responses).

The curve maximum represents the learning capacities. The slope of the curve evaluates the learning speed. The area under the curve (AUC) represents a good evaluation of all conditioning parameters. The maximum value of the area under the curve is 500, if the animals show 100% adequate responses as early as Day 0. In fact, an average area under the curve is calculated per day, what amounts to a maximum value of 100. In the absolute control animals, this average area under the curve is equal to 40+/−4.

2.6 Reagents

All reagents used are grade I reagents, and are provided by Aldrich (Saint Quentin Fallavier, France).

Dexamethasone 3H is provided by Amersham (England).

The specific agonist (RU 28362) is provided by Roussel.

2.7 Statistical Analysis

Results are expressed as an average with the standard error of mean (SEM) of the results obtained from ten rats par experimental group.

Following a ANOVA variance analysis, significant results are obtained by means of a Student's test.

The variability is calculated as a function of the least squares for each experimentation day. Significance is determined by a t-test.

3. Results

3.1 Gp120 Model

Sound Avoidance Conditioning by the Pole Climbing Test Method

The results are presented in Table I below. For each learning day, the values listed in the table correspond to the percentage of avoidance. The rat avoids getting the electric discharge by climbing the pole. On the first day, no avoidance is observed in any of the animal groups. The learning speed and capacities are significantly decreased in rats that receive Gp120. The treatment with Cinnamoyl-Gly-Phe-ProNH₂ and with tacrine (TAA) partly restores the learning parameters. This significant result is observed for the areas under the curve and for time on Day 2, 3, and 4.

| Treatment | Day 0 | Day 1 | Day 2 | Day 3 | Day 4 | AUC |
|---|---|---|---|---|---|---|
| without | 0 | 8 +/− 2 | 34 +/− 4 | 71 +/− 4 | 90 +/− 3 | 40.6 +/− 2.6 |
| GP120 | | | | | | |
| GP120 | 0 | 3 +/− 2 | 8 +/− 3 | 12 +/− 4 | 18 +/− 3 | 8.2 +/− 2.4 |
| GP120 + tripeptide | 0 | 5 +/− 2 | **18 +/− 2 | **32 +/− 2 | **43 +/− 3 | **19.6 +/− 1.8 |
| derivative | | | | | | |
| GP120 + tacrin | 0 | 5 +/− 2 | **18 +/− 2 | **31 +/− 3 | **42 +/− 4 | 19.4 +/− 2.2 |

**p > 0.01 in comparison with the groups without GP120 administration or with only GP120 administration

Neuronal Count

After the last learning session, the rats were sacrificed by means of decapitation, and the brain was isolated and frozen to −80° C. with liquid nitrogen. Cuts of a thickness of 50 μm were obtained in a “cryo-cut”, and the neurons are counted in the CA III layers of the hippocampus under a microscope. The results are expressed as percentage of the control group.

| | Percentage of hippocampal |
|---|---|
| Treatment | neurons |
| without Gp120 | 100 ± 3 |
| Control with Gp120 | 43 ± 4 |
| GP 120 + tripeptide | *54 ± 3 |
| derivative | |
| GP 120 + tacrine | 45 ± 3 |

*p > 0,05: Comparison between treated and non-treated animals

As is apparent from these results, administration of the tripeptide leads to an improvement of the learning capacities, similar to tacrine. However, in contrast to degeneration of the neurons.

3.2 Dexamethasone Model

Sound Conditioning

The learning capacity was studied as described above. The results are summarized in the following Table.

| Treatment | Day 0 | Day 1 | Day 2 | Day 3 | Day 4 | AUC |
|---|---|---|---|---|---|---|
| without | 0 | 8 +/− 2 | 36. +/− 4 | 71 +/− 4 | 90 +/− 3 | 40.6 +/− 2.6 |
| Dexam. | | | | | | |
| Dexam. | 0 | 5 +/− 2 | 16 +/− 4 | 45 +/− 5 | 54 +/− 5 | 24.0 +/− 1.6 |
| Dexam. + tripeptide | 0 | 8 +/− 3 | **28 +/− 3 | **68 +/− 3 | **77 +/− 3 | **36.2 +/− 1.2 |
| derivative | | | | | | |
| Dexam. + tacrine | 0 | 5 +/− 2 | 15 +/− 3 | 42 +/− 4 | 52 +/− 4 | 22.8 +/− 1.3 |

**p > 0.01 Comparison between treated and non-treated animals

Determination of the Corticoide Receptors

The results are summarized in the following Table.

| | Type I receptors in Femtomol/mg |
|---|---|
| Treatment | protein |
| without Dexam. | 120 ± 11 |
| Dexam. | 42 ± 7 |
| Dexam + tripeptide | **85 ± 9 |
| derivative | |
| Dexam. + tacrine | 46 ± 5 |

The administration of dexamethasone induces a very drastic decrease in the number of corticoide receptors in the hippocampus. For the dosage and the treatment time used in this experiment, a decrease in the order of 60% was found. The administration of the tripeptide to be used according to the present invention results in a significant increase of the number of receptors of hippocampus neurons. Tacrine does not show any effect under the same conditions.

This shows that the treatment with the tripeptide to be used according to the present invention considerably restores the learning capacity and the degeneration on the level of the hippocampus is decreased. On the other hand, tacrine, which inhibits the degradation of acetyl choline and improves the learning behaviour, as far as acetyl choline is related, does not show any effect in this model of corticoide degeneration.

3.3 Vincristine Model

The learning behaviour was studied as described above. The results are summarized in the following Table.

| | | | | | | AUC pro |
|---|---|---|---|---|---|---|
| Treatment | Day 0 | Day 1 | Day 2 | Day 3 | Day 4 | Tag |
| without | 0 | 8 +/− 2 | 34 +/− 4 | 71 +/− 4 | 90 +/− 3 | 40.6 +/− 2.6 |
| Vin. | | | | | | |
| Vin. | 0 | 2 +/− 1 | 7 +/− 3 | 19 +/− 3 | 29 +/− 4 | 11.4 +/− 1.1 |
| Vin. + tripeptide | 0 | 5 +/− 2 | **20 +/− 2 | **45 +/− 3 | **53 +/− 3 | **24.6 +/− 1.0 |
| derivative | | | | | | |
| Vin. + tacrine | 0 | 2 +/− 1 | 7 +/− 2 | 19 +/− 2 | 28 +/− 3 | 11.2 +/− 0.8 |

**p > 0.01 Comparison between treated and non-treated animals

These results show the advantageous effects of the substances to be used according to the present invention on the learning capacities. On the other hand, tacrine does not show an effect in this model.

1. A method for the treatment of neurodegenerative diseases comprising administering an effective amount of a compound of formula (I) to a human patient in need thereof: 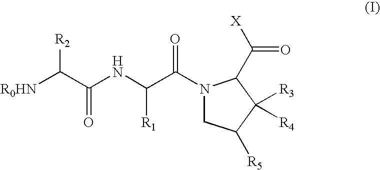
wherein X represents OH, (C₁₋₅)alkoxy, NH₂, NH—C₁₋₅-alkyl, or N(C₁₋₅alkyl)₂;
R₁ is a residue derived from any of the amino acid Phe, Tyr, or Trp each of which may optionally be substituted by a (C₁₋₅)alkoxy groups, a (C₁₋₅)alkyl group or a halogen atom, or a residue derived from Ala, Val, Leu, or Ile;
R₂ is a residue which is derived from any of the amino acids Gly, Ala, Ile, Val, Ser, Thr, His, Arg, Lys, Glu, Gln, Asp, Leu or Asn;
R₃-and R₄ independently represent H, OH, (C₁₋₅)alkyl, or (C₁₋₅)alkoxy, provided that R₃ and R₄ are not both OH or (C₁₋₅)alkoxy;
R₅ represents H, OH, (C₁₋₅)alkyl or (C₁₋₅)alkoxy;
and wherein R₀ represents a group of the formula 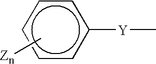
wherein Y represents —CO—, —CH₂CO—, —CH₂CH₂CO—, —CH₂CH₂CH₂CO—, —CH═CH—CO or —OCH₂CO—, and wherein Z represents a halogen atom, a trifluormethyl group, (C₁₋₄)alkoxy group, (C₁₋₄)alkyl group; or wherein two neighbouring substituents may form a (C₁₋₃) alkylendioxy group; and wherein n is 0 or an integer of from 1 to 5;
or pharmaceutically acceptable salts thereof;
or a pharmaceutically acceptable salt thereof.

2. The method according to claim 1, wherein R₁ is a residue derived from any of the amino acids Phe, Tyr, Trp, each of which may optionally be substituted by a (C₁₋₅)alkoxy group, a (C₁₋₅)alkyl group or a halogen atom, or a residue derived from the amino acid Ile.

3. The method according to claim 2, wherein R₁ is a residue derived from Phe which may optionally be substituted by a (C₁₋₅)alkoxy groups, a (C₁₋₅)alkyl group or a halogen atom.

4. The method according to claim 1, wherein X is (C₁₋₅)alkoxy, NH₂, NH—C₁₋₅-alkyl, or N(C₁₋₅alkyl)₂.

5. The method according to claim 1, wherein R₂ is a residue derived from the amino acid Gly or Ile.

6. The method according to claim 1, wherein R₀ is a cinnamoyl moiety.

7. The method according to claim 1, wherein the compound of formula (I) is a cinnamoyl-glycyl-L-phenylalanyl-L-prolinamide, cinnamoyl-isoleucyl-phenylalanyl-L-proline ethylamide, cinnamoyl-isoleucyl-isoleucyl-prolineamide, or a pharmaceutically acceptable salt thereof.
